(19) United States
(12) Patent Application Publication
(10) Pub. No.: US 2026/0365543 A1
(43) Pub. Date: Nov. 19, 2026

(54) WATER DISPENSING DEVICE

(71) Applicant: LG ELECTRONICS INC., Seoul (KR)

(72) Inventors: Chulwoo PARK, Seoul (KR); Yumin KIM, Seoul (KR); Ilna SON, Seoul (KR); Sangjoon LEE, Seoul (KR)

(21) Appl. No.: 19/148,399

(22) PCT Filed: Jun. 29, 2023
(86) PCT No.: PCT/KR2023/009083

§ 371 Date: Jul. 16, 2025

(30) Foreign Application Priority Data
Jan. 18, 2023 (KR) 10-2023-0007294

(51) Int. Cl. C02F 1/00; C02F 1/02

(52) U.S. Cl. CPC C02F 1/001; C02F 1/02; C02F 2201/005; C02F 2209/003; C02F 2209/11; C02F 2303/04; C02F 2303/24; C02F 2307/10

(57) ABSTRACT

A water dispensing apparatus according to an embodiment of the present disclosure includes: a water supply passage through which raw water, supplied from a water source, flows; a filter configured to generate purified water by filtering the raw water supplied through the water supply passage; a purified water passage through which the purified water, having passed through the filter, flows; and a water quality measurement unit connected to the purified water passage and configured to measure water quality of the purified water, wherein the water quality measurement unit includes a turbidity sensor module configured to measure turbidity of water, and a virtual impactor disposed at a front end of the turbidity sensor module.

### TECHNICAL FIELD

The present description relates to a water dispensing apparatus and a method of operating the same, and more particularly to a water dispensing apparatus including a sensor capable of determining water quality, and a method of operating the same.

### BACKGROUND ART

A water dispensing apparatus is an apparatus for supplying water and dispensing a desired amount of water at a desired temperature according to a user's operation. The water dispensing apparatus may be applied to various fields, but may be typically applied to refrigerators and water purifiers. Particularly, the water dispensing apparatus provided in the refrigerator and the water purifier may be configured to automatically supply a set amount of water according to a user's operation. Recently, water dispensing apparatuses capable of supplying not only purified water but also cold water and hot water have been developed.

For example, a water purifier is connected to a water source, such as a water faucet and the like, to receive raw water, and is configured to remove floating substances and harmful substances contained in the raw water by using a filter and to purify and dispense a desired amount of water according to a user's operation. Various purifiers are on the market, including a purifier capable of not only purifying water, but also heating or cooling purified water to supply hot water or cold water. Further, purifiers having a compact size and capable of being installed in various installation environments are being developed these days.

If the water dispensing apparatus is used for a long time, microorganisms and the like may proliferate in pipes, valves, and water outlets, or the apparatus may be contaminated, and if a filter replacement period has elapsed, floating substances and harmful substances contained in raw water may not be removed. Accordingly, it is important to measure water quality accurately and to manage the water dispensing apparatus hygienically, and purified water quality performance should be managed as well.

Korean Laid-open Patent Publication No. 10-2017-0005264 as related art discloses a water treatment apparatus including a water quality measurement unit configured to measure water quality of water stored in a purified water tank and to generate water quality information, a circulation passage providing a path for resupplying the purified water stored in the purified water tank to a filter unit, and a controller configured to drain the purified water stored in the purified water tank or resupply the water to the filter unit through the circulation passage based on the water quality information.

### DISCLOSURE OF INVENTION

### Technical Problem

It is an objective of the present disclosure to provide a water dispensing apparatus capable of measuring water quality more accurately.

It is another objective of the present disclosure to provide a water dispensing apparatus capable of efficiently sterilizing a passage within a short period of time.

It is yet another objective of the present disclosure to provide a water dispensing apparatus capable of managing a passage hygienically by automatically detecting abnormal water quality and performing sterilization.

It is yet another objective of the present disclosure to provide a water dispensing apparatus capable of selectively sterilizing a passage in response to a water quality measurement result.

It is yet another objective of the present disclosure to provide a water dispensing apparatus capable of accurately measuring water quality of raw water and purified water.

It is yet another objective of the present disclosure to provide a water dispensing apparatus capable of improving sensing accuracy and efficiency by a rinse operation and water pipe configuration for sharing a sensor.

### Solution to Problem

In accordance with an aspect of the present disclosure, the above and other objectives can be accomplished by providing a water dispensing apparatus including: a water supply passage through which raw water, supplied from a water source, flows; a filter configured to generate purified water by filtering the raw water supplied through the water supply passage; a purified water passage through which the purified water, having passed through the filter, flows; and a water quality measurement unit connected to the purified water passage and configured to measure water quality of the purified water, wherein the water quality measurement unit includes a turbidity sensor module configured to measure turbidity of water, and a virtual impactor disposed at a front end of the turbidity sensor module.

The virtual impactor may be configured to reduce a flow rate of water flowing into the turbidity sensor module, by passing only some of water that flows into the water quality measurement unit.

The virtual impactor may be configured to classify by size contaminants contained in the water flowing into the water quality measurement unit, to increase turbidity of the water flowing into the turbidity sensor module.

The water dispensing apparatus may further include: a drain passage through which the raw water or the purified water is drained; an impactor drain passage which is connected to the drain passage, and through which water not introduced by the virtual impactor into the turbidity sensor module is discharged; and an impactor discharge valve disposed in the impactor drain passage.

The water dispensing apparatus may further include: a water outlet through which the purified water is discharged; and a water discharge passage guiding the purified water to the water outlet, wherein the drain passage may be branched from the water discharge passage between the water quality measurement unit and the water outlet.

The water dispensing apparatus may further include a water discharge valve selectively supplying the raw water or the purified water to the water discharge passage and the drain passage.

The virtual impactor may be provided as a multi-stage impactor including impactors that separate particles of different sizes.

The virtual impactor may further include: a first stage impactor configured to separate a first particle which is a largest particle; a second stage impactor configured to separate a second particle which is smaller than the first particle and a third particle which is a smallest particle; a first impactor passage through which the first particle separated by the first stage impactor flows; a second impactor passage through which the second particle separated by the second stage impactor flows; and a third impactor passage through which the third particle separated by the second stage impactor flows.

The virtual impactor may further include: a first impactor valve disposed between the first impactor passage and the impactor drain passage; a second impactor valve disposed between the first impactor passage and the turbidity sensor module; a third impactor valve disposed between the second impactor passage and the impactor drain passage; a fourth impactor valve disposed between the second impactor passage and the turbidity sensor module; a fifth impactor valve disposed between the third impactor passage and the impactor drain passage; and a sixth impactor valve disposed between the third impactor passage and the turbidity sensor module.

The water dispensing apparatus may further include: a sterilization passage having a first side branched from the water supply passage and a second side connected to the filter side; and a sterilization module provided in the sterilization passage and configured to heat water passing through the sterilization passage, wherein a sterilization operation is performed for each passage region by controlling the hot water, discharged from the sterilization module, to flow to different passage regions based on water quality data measured by the water quality measurement unit.

In response to water quality data measured for the third particle by the water quality measurement unit being greater than or equal to a first reference value, the water dispensing apparatus may be configured to sterilize the purified water passage with the hot water discharged from the sterilization module.

The water dispensing apparatus may further include: a hot water passage having one side branched from the purified water passage; a hot water module provided in the hot water passage and configured to heat purified water passing through the hot water passage; a cold water passage having one side branched from the purified water passage; and a cold water module provided in the cold water passage and configured to cool purified water passing through the cold water passage, wherein in response to water quality data measured for the third particle by the water quality measurement unit being greater than or equal to a second reference value that is greater than the first reference value, the water dispensing apparatus may be configured to sterilize the purified water passage and the cold water passage with the hot water discharged from the sterilization module.

In response to water quality data measured for the third particle by the water quality measurement unit being greater than or equal to a third reference value that is greater than the second reference value, the water dispensing apparatus may be configured to sterilize an entire passage with the hot water discharged from the sterilization module.

In response to water quality data measured for the third particle by the water quality measurement unit being greater than a second reference value that is greater than the first reference value, the water dispensing apparatus may be configured to sterilize the purified water passage and the water discharge passage with the hot water discharged from the sterilization module.

In response to the water quality data measured for the second particle by the water quality measurement unit being greater than or equal to the first reference value, the water dispensing apparatus may be configured to sterilize the entire passage with the hot water discharged from the sterilization module.

The water dispensing apparatus may further include an output unit which, in response to water quality data measured for the first particle by the water quality measurement unit being greater than or equal to the first reference value, is configured to display abnormal water quality information.

The water dispensing apparatus may further include a sensing passage which is branched from the water supply passage, and through which the raw water flows, wherein the water quality measurement unit may be configured to measure water quality of the purified water in response to the purified water being introduced through the purified water passage, and configured to measure water quality of the raw water in response to the raw water being introduced through the sensing passage.

Upon measuring the water quality of the raw water, the water quality measurement unit is configured to measure the water quality of the purified water after performing a rinse operation in which the purified water passes through the water quality measurement unit one or more times.

The water dispensing apparatus may further include: a switching valve configured to supply the raw water to the water supply passage or the sterilization passage; and a sensing valve configured to open and close the sensing passage.

### Advantageous Effects of Disclosure

According to at least one of the embodiments of the present disclosure, water quality may be measured more accurately.

According to at least one of the embodiments of the present disclosure, a passage may be sterilized efficiently within a short period of time.

According to at least one of the embodiments of the present disclosure, a passage may be managed hygienically by automatically detecting abnormal water quality and performing sterilization.

According to at least one of the embodiments of the present disclosure, a passage may be sterilized selectively in response to a water quality measurement result.

According to at least one of the embodiments of the present disclosure, sensing accuracy and efficiency may be improved by a rinse operation and water pipe configuration for sharing a sensor.

Meanwhile, various other effects will be directly or implicitly disclosed in the following detailed description of embodiments of the present disclosure.

### BRIEF DESCRIPTION OF DRAWINGS

FIG. 1 is a block diagram of main components of a water dispensing apparatus according to an embodiment of the present disclosure.

FIG. 2 is a conceptual diagram illustrating a water dispensing apparatus according to an embodiment of the present disclosure.

FIGS. 3 to 6 are diagrams referred to in the description of operation of the water dispensing apparatus of FIG. 2.

FIG. 7 is a conceptual diagram illustrating a water dispensing apparatus according to an embodiment of the present disclosure.

FIG. 8 is a diagram referred to in the description of optical turbidity sensing using light.

FIGS. 9 to 11 are diagrams referred to in the description of a virtual impactor according to an embodiment of the present disclosure.

FIG. 12 is a diagram referred to in the description of hygiene management standards for a water dispensing apparatus according to an embodiment of the present disclosure.

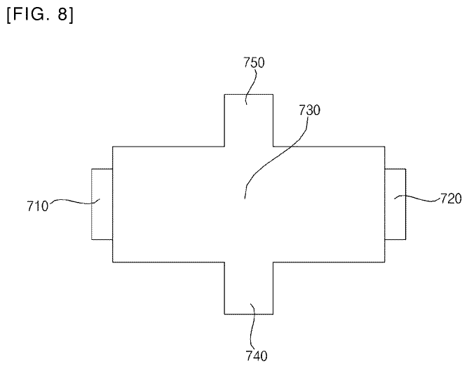

FIGS. 8 to 12 are diagrams referred to in the description of a turbidity sensor module according to various embodiments of the present disclosure.

FIGS. 13 to 15 are diagrams referred to in the description of sterilizing a water dispensing apparatus according to an embodiment of the present disclosure.

FIGS. 16 and 17 are diagrams referred to in the description of distribution of measured turbidity values.

### MODE OF DISCLOSURE

Hereinafter, embodiments of the present disclosure will be described in detail with reference to the accompanying drawings. However, it is understood that the present disclosure is not limited to these embodiments and may be modified in various forms.

In the drawings, in order to clearly and briefly describe embodiments of the present disclosure, the illustration of parts irrelevant to the description is omitted, and the same reference numerals are used for the same or extremely similar parts throughout the specification.

Hereinafter, the suffixes “module” and “unit” of elements herein are used for convenience of description and thus may be used interchangeably and do not have any distinguishable meanings or functions. Thus, the terms “module” and “unit” may be interchangeably used.

It will be understood that, although the terms “first”, “second”, etc. may be used herein to describe various elements, these elements should not be limited by these terms. These terms are only used to distinguish one element from another element.

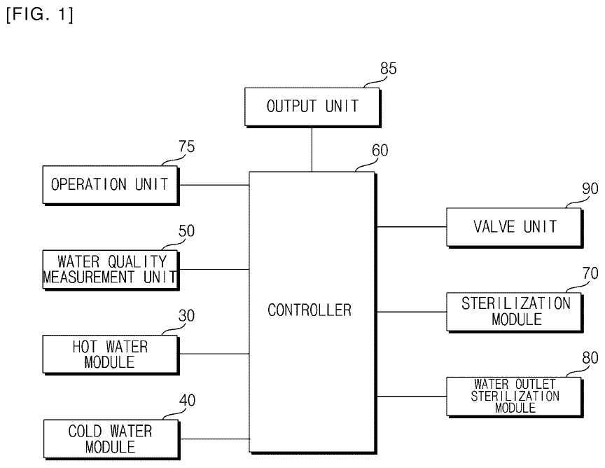

FIG. 1 is a block diagram of main components of a water dispensing apparatus according to an embodiment of the present disclosure.

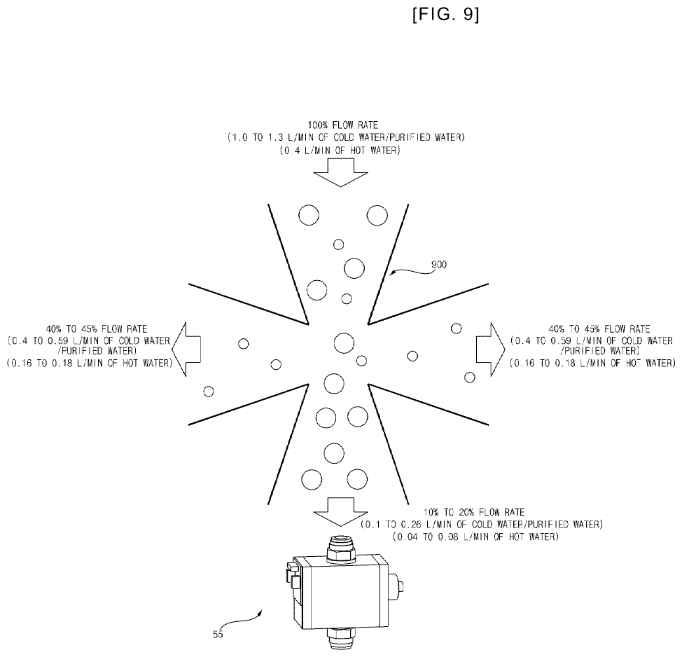

Referring to FIG. 1, the water dispensing apparatus according to an embodiment of the present disclosure includes a water quality measurement unit 50. The water quality measurement unit 50 may include a turbidity sensor module 55 (see FIG. 9). According to an embodiment of the present disclosure, turbidity (contamination level) may be detected in an optical sensing manner. A transmitted light method and a scattered light method are used as an optical sensing method for measuring turbidity. The transmitted light method senses turbidity by emitting light to a fluid, receiving light having passed through the fluid, and processing data. The scattered light method senses turbidity by receiving scattered light and converting it into data, and the scattered light method is classified according to a method of generating scattered light and a method of processing the received light data. The turbidity sensor module 800 according to an embodiment of the present disclosure may emit light to some of raw water or purified water, and may sense turbidity based on a received scattered light pattern. According to an embodiment of the present disclosure, particles and microorganisms are separated by patterning the movement and intensity of light scattered by microorganisms, and big data processing is performed to distinguish between types of indicator microorganisms according to water quality/sanitation standards. In addition, detected microbial concentration values and safety indicators are provided on a display so as to be intuitively identified by a user.

In addition, the water quality measurement unit 50 may employ a water quality measurement sensor, such as a turbidity sensor, a microbial sensor, a TDS sensor, etc., in order to detect contamination of water in a passage. The water quality measurement unit 50 may include at least one of a turbidity sensor, a microbial detection sensor, a chlorine sensor, a Total Dissolved Solids (TDS) sensor, and a Biochemical Oxygen Demand (BOD) sensor, and may measure at least one of the turbidity, microorganisms, residual chlorine, Total Dissolved Solids (TDS), and dissolved oxygen of introduced water. At least one of the sensors included in the water quality measurement unit 50 may be a shared sensor that measures the water quality of both raw water and purified water.

In the present disclosure, the flow of purified water or raw water into the water quality measurement unit 50 does not only mean that purified water or raw water flows into the water quality measurement unit 50. For example, some of the purified water or raw water may be sampled for measuring water quality in the water quality measurement unit 50, and then may be discharged after the measurement. In addition, at least some of the sensors included in the water quality measurement unit 50 may measure water quality of a liquid that flows. In this case, the flow of purified water or raw water into the water quality measurement unit 50 may mean that at least some of the purified water or raw water passes through a sensing capable region of the water quality measurement unit 50.

For example, in the case where the turbidity sensor module 800 includes an inner chamber, and when water flows into the inner chamber connected to a passage 20 and fills the chamber, the turbidity sensor module 800 may measure water quality by emitting light to the water that fills the chamber and receiving a scattered light pattern. In addition, the water inside the inner chamber may be discharged after measuring the water quality. Alternatively, a light source and a light receiver of the turbidity sensor module 800 may be positioned in a specific passage section (e.g., a section after a purified water passage 20 and a sensing passage 12 are joined), to emit light to the purified water or raw water that passes through the specific passage section and to receive a scattered light pattern.

The water dispensing apparatus according to an embodiment of the present disclosure may measure water quality of raw water and purified water by using a shared sensor for the same measurement items, such as turbidity and the like. The turbidity sensor module may measure the turbidity of raw water and the turbidity of purified water and transmit the sensing data to a controller 60. The controller 60 may control other components of the water dispensing apparatus based on the sensing data of the water quality measurement unit 50, such as a turbidity sensor and the like.

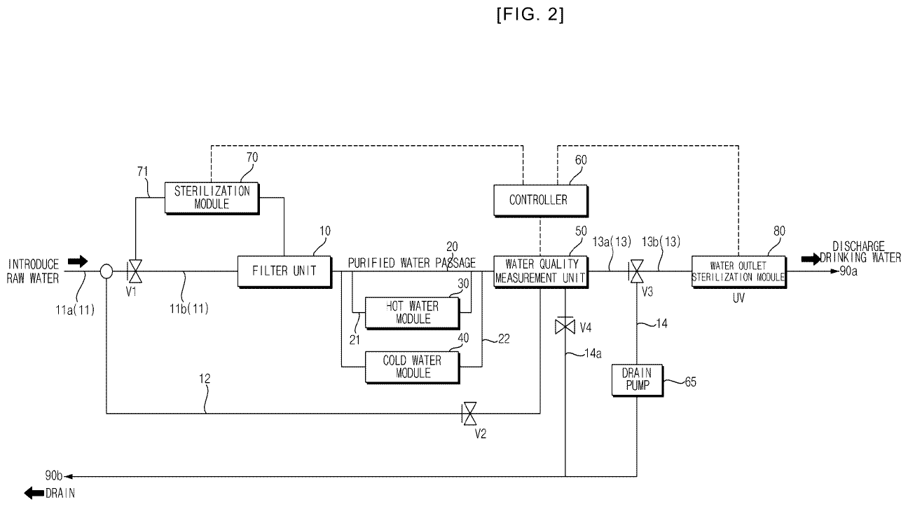

The water dispensing apparatus includes a filter 10 (see FIG. 2, etc.) configured to generate purified water by filtering raw water supplied from a water source. The filter 10 is provided to purify the supplied raw water and filter out various impurities and harmful substances contained in the raw water. One or more filters 10 are provided, and in the case where a plurality of filters are provided, a combination of filters having various functions may be used. For example, three filters 10 may be provided, including a pre-carbon filter, post-carbon filter, and a membrane filter or a hollow fiber membrane filter that is disposed between the pre-carbon filter and post-carbon filter. Alternatively, the filter 10 may include a pre-carbon filter and a UF composite filter.

Purified water purified by the filter 10 flows to a storage tank or a passage 20 (see FIG. 2, etc.). As water stored in the storage tank provides an environment suitable for propagation of microorganisms over time, it is more preferable that the water immediately flows through the passage 20. The purified water having passed through the filter 10 flows to the purified water passage 20.

In addition, the water dispensing apparatus includes a valve unit 90 including valves for controlling the flow of water. The valve unit 90 may include a plurality of valves V1, V2, V3, V4, etc., which will be described later.

When the water quality measurement unit 50 measures the water quality of the raw water, the controller 60 may control the valve unit 90 and the like so that a rinse operation is performed in which the purified water passes through the water quality measurement unit 50 one or more times.

The water quality measurement unit 50 measures the water quality of the purified water after performing the rinse operation, thereby minimizing the effect of raw water on the water quality measurement of the purified water. Accordingly, by using only one water quality sensor of the same type, it is possible to efficiently and accurately sense each of the raw water and the purified water.

When tap water and purified water are discharged, they normally have low water contamination levels, and in order to measure low contamination concentrations, it is important to minimize the deviation of measurements. According to an embodiment of the present disclosure, by providing and controlling passages capable of simultaneously measuring the raw water and discharged water using one sensor, the increase in material costs may be minimized and the product may be manufactured in a compact size, compared to techniques that separately measure raw water and discharged water.

The water quality of tap water (raw water) and water quality of a water purifier (purified water) mostly fall into a low concentration range, and in order to clearly show a performance difference between the raw water and discharge water, it is most important to minimize the measurement deviation between devices by comparing using one sensor.

In addition, the water dispensing apparatus may include a hot water module 30 and a cold water module 40 configured to supply hot water/cold water. The hot water module 30 heats purified water, and then discharges the water toward a water outlet 90a (see FIG. 2, etc.). The cold water module 40 cools the purified water, and then discharges the water toward the water outlet 90a.

In addition, the water dispensing apparatus further includes an operation unit 75 and an output unit 85.

The operation unit 75 may receive user input and include one or more buttons. For example, the operation unit 75 may be provided as a touch panel and may include a capacity button for selecting a capacity of water to be discharged, a hot water button for selecting hot water and temperature of the hot water to be discharged, a purified water button for selecting purified water, a cold water button for selecting cold water, and other function buttons.

The output unit 85 may include a display device, such as a display (not shown) or a light emitting diode (LED) (not shown), and the like. For example, the output unit 85 may display information such as the operating state of the water dispensing apparatus, operating state related to error occurrence or the like, or water contamination level, etc.

The output unit 85 may include an audio device, such as a speaker (not shown), a buzzer (not shown), and the like. For example, the output unit 85 may output a sound effect for the operating state of the water dispensing apparatus and output a predetermined warning sound when an error occurs.

In addition, the water dispensing apparatus may further include modules for sanitation. For example, the water dispensing apparatus includes a sterilization module 70 using high-temperature or hot water. In addition, the water dispensing apparatus includes a water outlet sterilization module 80 for sterilization on the side of the water outlet 90a where there is a high possibility of contamination.

The sterilization module 70 instantly heats water to a high temperature to sterilize bacteria growing in water. In addition, the controller 60 may also operate the sterilization module 70 so that sterilized water (hot water) discharged from the sterilization module 70 may circulate through another passage to sterilize the passage. Based on water quality data measured by the water quality measurement unit 50, the controller 60 may control the hot water, discharged from the sterilization module 70, to flow to different passage regions to perform a sterilization operation for each passage region.

The water outlet sterilization module 80 may remove bacteria or viruses by emitting ultraviolet (UV) rays toward the water outlet 90a. The water outlet sterilization module 80 may include at least one Ultraviolet rays (UV) lamp or at least one Ultraviolet rays Light Emitting Diode (UV LED).

The water outlet sterilization module 80 may be periodically driven under control of the controller 60. Alternatively, the water outlet sterilization module 80 may be driven during a predetermined period of time before water is discharged. More preferably, based on water quality data measured by the water quality measurement unit 50, the controller 60 may drive the water output sterilization module 80 only when it is required, thereby improving efficiency. For example, the controller 60 may control the water outlet sterilization module 80 based on a result of purified water quality measurement.

The controller 60 may be connected to each component provided in the water dispensing apparatus. For example, the controller 60 may transmit and/or receive signals with the respective components provided in the water dispensing apparatus and may control the overall operation of the respective components.

The controller 60 may include at least one processor, and may control the overall operation of the water dispensing apparatus by using the processor included therein. Here, the processor may be a general processor such as a central processing unit (CPU). Obviously, the processor may be a dedicated device such as an ASIC or another hardware-based processor.

The controller 60 may perform various operations based on data received from the water quality measurement unit 50 including various sensors such as the turbidity sensor 800 and the like. In addition, the controller 60 may store data received from the water quality measurement unit 50 in a memory (not shown).

The water quality measurement unit 50 may measure water quality and output it to the controller 60. The controller 60 may perform a feedback operation in response to the received raw water and/or purified water quality measurement data. Alternatively, the water quality measurement unit 50 may directly determine a contamination level and transmit it to the controller 60, and the controller 60 may control other components to perform appropriate feedback operations based on the received contamination level.

By identifying the contamination state of raw water and/or purified water, the controller 60 may control the output unit 85 to provide a user with cleaning alarm or information about filter replacement period.

In addition, the controller 60 may detect odor that may occur based on a contamination level of raw water and/or purified water, and may perform an automatic cleaning/sterilization logic by operating the sterilization module 70 and the water outlet sterilization module 80 before a customer recognizes it. Accordingly, it is possible to improve user convenience and hygiene for non-professional users.

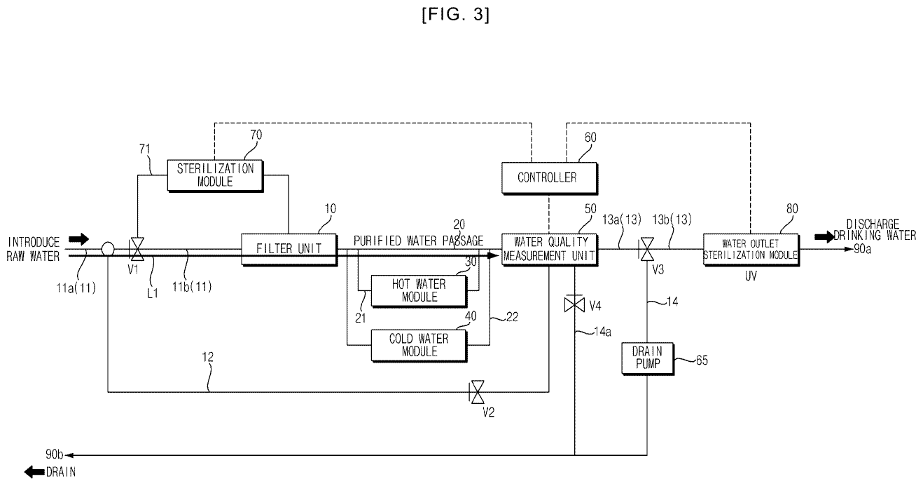

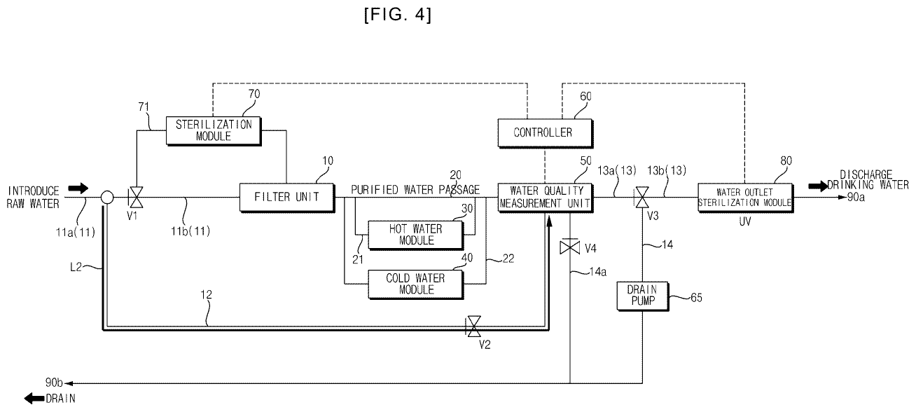

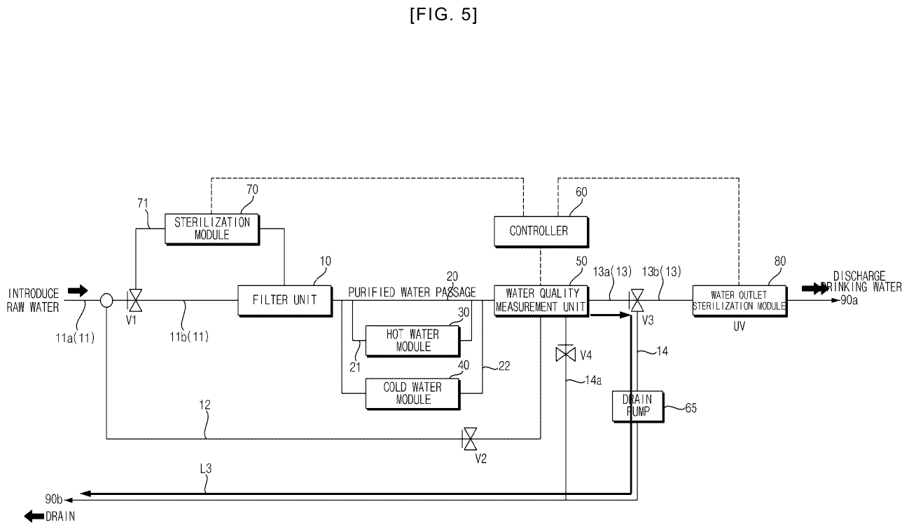

FIG. 2 is a conceptual diagram illustrating a water dispensing apparatus according to an embodiment of the present disclosure, and FIGS. 3 to 6 are diagrams referred to in the description of operation of the water dispensing apparatus of FIG. 2.

Referring to FIG. 2, the water dispensing apparatus includes a water supply passage 11 through which raw water supplied from a water source flows, and a filter unit 10 configured to generate purified water by filtering the raw water supplied through the water supply passage 11.

The purified water having passed through the filter unit 10 may flow toward the water outlet 90a through the purified water passage 20. The purified water having passed through the filter unit 10 may flow into the water quality measurement unit 50. When the purified water is introduced, the water quality measurement unit 50 may measure water quality of the purified water.

In addition, a sensing passage 12 may be branched from the water supply passage 11 such that the raw water may directly flow into the water quality measurement unit 50 through the sensing passage 12. When the raw water is introduced, the water quality measurement unit 50 may measure water quality of the raw water.

According to an embodiment of the present disclosure, the filter unit 10 and a water supply valve V1 configured to control the supply of water toward the purified water passage 20 may be disposed in the water supply passage 11. The water supply valve V1 may open and close the purified water passage 20. When the water supply valve V1 opens, the raw water is purified by passing through the filter unit 10 along a first line L1 of FIG. 3, and the purified water may flow into the water quality measurement unit 50 through the purified water passage 20.

Meanwhile, the water supply passage 11 may include a first water supply passage 11a connecting the water source and the water supply valve V1, and a second water supply passage 11b connecting the water supply valve V1 and the filter 10.

In addition, the sensing passage 12 may have a first end connected to the first water supply passage 11a, and a second end connected to the water quality measurement unit 50. A valve V2 configured to open and close the sensing passage 12 may be disposed in the sensing passage 12. When a sensing valve V2 opens, the raw water may directly flow into the water quality measurement unit 50 through the sensing passage 12 along a second line L2 of FIG. 4.

Referring to FIG. 2, the water dispensing apparatus may further include a water outlet 90a through which the purified water is discharged, a water discharge passage 13 guiding the purified water to the water outlet 90a, a drain passage 14 which is branched from the water discharge passage 13 between the water quality measurement unit 50 and the water outlet 90a and through which the raw water or the purified water is drained, and a water discharge valve V3 selectively supplying the raw water or the purified water to the water discharge passage 13 and the drain passage 14.

The water discharge valve V3 may divert water, for which water quality measurement is completed, to a drain 90b and the water outlet 90a under control of the controller 60. When a drain operation is performed, the water, for which the water quality measurement is completed, flows to the drain passage 14. By draining and cleaning the raw water, it is possible to prevent the raw water from being discharged as drinking water.

Meanwhile, the water discharge passage 13 may include a first water discharge passage 13a connecting the water quality measurement unit 50 and the water discharge valve V3, and a second water discharge passage 13b connecting the water discharge valve V3 and the water outlet 90a.

Referring to FIG. 2, the water dispending apparatus may include a water supply passage 11 through which raw water supplied from a water source flows, a filter unit 10 configured to generate purified water by filtering the raw water supplied through the water supply passage 11, a purified water passage 20 through which the purified water having passed through the filter unit 10 flows, and a water quality measurement unit 50 connected to the purified water passage 20 and configured to measure water quality of the purified water. In addition, the water quality measurement unit 50 includes a turbidity sensor module 55 (see FIG. 9, etc.) configured to measure turbidity of water, and a virtual impactor 900 (see FIG. 9, etc.) disposed at a front end of the turbidity sensor module 55.

In the case of a turbidity sensor, the accuracy and reliability of measurement values increase as a flow rate decreases. In addition, the accuracy and reliability of measurement values increase as the turbidity concentration increases. The virtual impactor 900 may concentrate submersible particles by using a difference in inertial force according to a particle size. The water dispensing apparatus according to an embodiment of the present disclosure may include the virtual impactor 900 placed at the front end of the turbidity sensor module 55, thereby increasing the turbidity concentration and reducing the measured flow rate to increase the accuracy and reliability of the measured turbidity values.

Referring to FIG. 2, the water dispensing apparatus may further include a drain passage 14 through which the raw water or the purified water is drained, an impactor drain passage 14a which is connected to the drain passage 14 and through which water not flowing from the virtual impactor 900 into the turbidity sensor module 55 is discharged, and an impactor discharge valve V4 disposed in the impactor drain passage 14a.

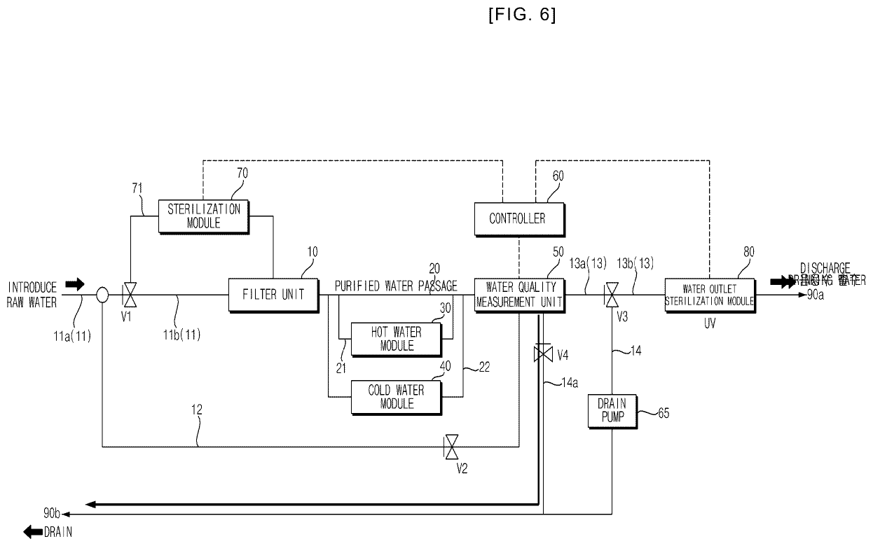

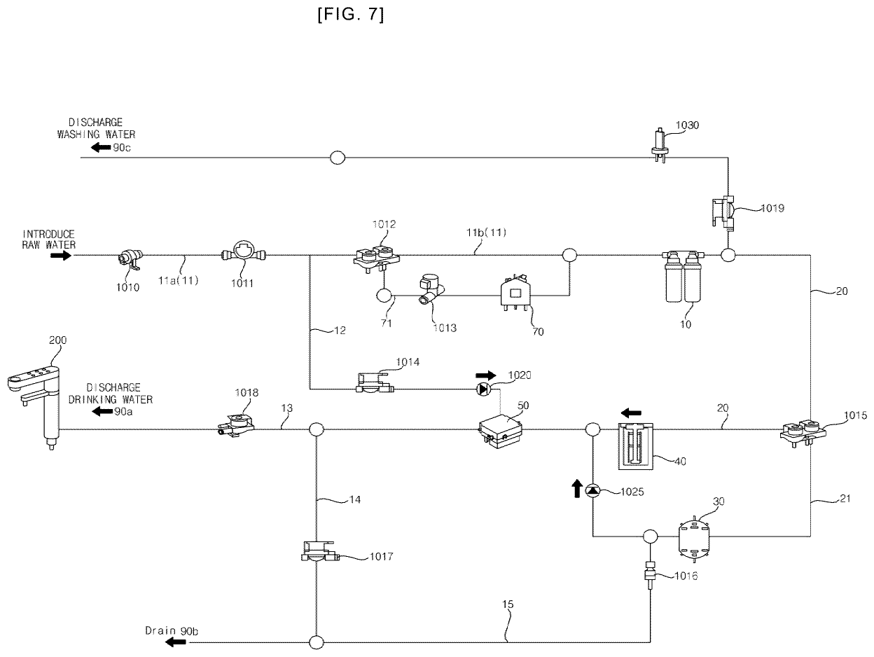

The impactor discharge valve V4 is a drain valve for discharging residual water generated when the virtual impactor 900 is activated. When the virtual impactor 900 is activated, water filtered by the virtual impactor 900 may flow to the drain passage 14 along a fourth line L4 in FIG. 6 to be discharged to the outside. An example of improving turbidity sensing accuracy using the virtual impactor 900 and operation of the water dispensing apparatus based on the sensed turbidity data will be described in detail with reference to FIGS. 8 to 17.

Meanwhile, the water outlet sterilization module 80 configured to emit UV rays to the water outlet 90a is disposed on the side of the water outlet 90a through which the purified water is discharged. The water outlet sterilization module 80 may sterilize the space of the water outlet and residual water. The controller 60 may operate the water outlet sterilization module 80 for a predetermined period of time based on the water quality data measured by the water quality measurement unit 50.

Referring to FIG. 2, a sterilization passage 71 may have a first side branched from the water supply passage 11 and a second side connected to the side of the filter 10, and the sterilization module 70 configured to heat water passing through the sterilization passage 71 is disposed in the sterilization passage 71. The controller 60 may operate the sterilization module 70 for a predetermined period of time based on the water quality data measured by the water quality measurement unit 50.

According to an embodiment of the present disclosure, the water supply valve V1 may be a switching valve selectively supplying the raw water to the water supply passage 11 or the sterilization passage 71.

Referring to FIG. 2, the water dispensing apparatus may further include a hot water passage 21 having one side branched from the purified water passage 20, a hot water module 30 provided in the hot water passage 21 and configured to heat purified water passing through the hot water passage 21, a cold water passage 22 having one side branched from the purified water passage 20, a cold water module 40 provided in the cold water passage 22 and configured to cool purified water passing through the cold water passage 22.

Referring to FIG. 2, the hot water passage 21 and the cold water passage 22 may join again the purified water passage 2. Alternatively, the hot water passage 21 and the cold water passage 22 may join again the water supply passage 13.

According to an embodiment of the present disclosure, a drain pump 65 may be disposed in the drain passage 14. By operating the drain pump 65 after the water quality is measured, the water, for which water quality measurement is performed, may be drained to the outside more rapidly at a faster rate.

In addition, the drain pump 65 may operate during a sterilization operation performed for each passage section. Accordingly, high-temperature or hot water after sterilization may be discharged more rapidly to the outside. Particularly, when the water discharge passage 13 connected to a cork on the side of the water outlet 90a is sterilized, some of the hot water is discharged toward the water outlet 90a, but a large amount of hot water may be discharged toward the drain 90b. Accordingly, it is possible to prevent safety accidents that may occur when a large amount of hot water is discharged, user discomfort, and inconvenience of a user having to deal with a large amount of hot water.

Meanwhile, when the purified water is introduced through the purified water passage 20, the water quality measurement unit 50 may measure water quality of the purified water, and when the raw water is introduced through the sensing passage 12, the water quality measurement unit 50 may measure water quality of the raw water.

Upon measuring the water quality of the raw water, the water quality measurement unit 50 may measure the water quality of the purified water after performing a rinse operation in which the purified water passes through the water quality measurement unit 50 one or more times. As described above, sensing accuracy and efficiency can be improved by a rinse operation and water pipe configuration for sharing a sensor.

FIG. 7 is a conceptual diagram illustrating a water dispensing apparatus according to an embodiment of the present disclosure.

The water dispensing apparatus according to an embodiment of the present disclosure may correspond to various water treatment devices and purification devices, such as purifiers, refrigerators, etc., in which water is introduced from an external source and the introduced water is purified, and then is discharged.

For example, the water dispensing apparatus may be provided as an under sink type water purifier, with at least a portion thereof being disposed in a space under a kitchen sink.

Referring to FIG. 7, the water dispensing apparatus according to an embodiment of the present disclosure may include a water discharge part 200 having at least a portion exposed to the outside of the sink, and a remaining body part installed inside the sink.

The water dispensing apparatus includes a water supply passage 11 guiding raw water supplied from the outside to the inside, a filter 10 configured to purify the raw water supplied along the water supply passage 11 into purified water, and a purified water passage 20 through which purified water, having passed through the filter 10, flows toward the water discharge part 200.

Meanwhile, the water supply passage 11 connects an external water source and the filter 10. Raw water, supplied from an external water source, may be supplied to the filter 10 through the water supply passage 11.

The water (raw water) supplied to the filter 10 may pass through the filter 10 to be purified into purified water. At least one filter 10 may be provided. For example, a plurality of filters 10 may be provided. Accordingly, water having passed through the water supply passage 11 may be purified into cleaner water by passing through the plurality of filters 10.

In addition, the purified water having passed through the filter 10 may flow toward the water discharge part 200, which is exposed to the outside of the sink 10, through the purified water passage 20.

To this end, a first end of the purified water passage 20 may be connected to the filter 10, and a second end thereof may be connected to the water discharge part 200. Meanwhile, at least one of the cold water passage 22, the hot water passage 21, and a washing water passage 90c may be branched from the purified water passage 20.

In FIG. 6, an example is illustrated in which the cold water passage 22 is integrated into the purified water passage 20, and the hot water passage 21 and the washing water passage 90c are branched from the purified water passage 20.

One end of the purified water passage 20 is connected to the filter 10, and water having passed through the filter 10 flows toward the water discharge part 200 through the connected water discharge passage 13. The water discharge passage 200 includes the water outlet 90a, and may dispense purified water and the like.

Water may pass through a washing water module 1030 provided in the washing water passage 90c, to be supplied in the state of sterilized water toward a washing water outlet. In the case where the water discharge part 200 includes a plurality of water outlets, the washing water outlet may also be formed in the water discharge part 200 in some examples.

Meanwhile, a decompression valve 1010 for adjusting a flow rate of water supplied to the filter 10 may be installed in the water supply passage 11.

In addition, at least one of a flow rate sensor 1011 for detecting a flow rate of water, a feed valve 35 for adjusting a flow rate of water or regulating the flow of water, or a flow speed sensor (not shown) for detecting a flow speed of the water may be installed in the water supply passage 11 or the purified water passage 20.

In addition, an opening/closing valve for regulating a flow of water in each passage may be installed separately in the purified water passage 20, the hot water passage 21, and the washing water passage 90c. For example, a washing water valve 1019 may be disposed in the washing water passage 90c.

Alternatively, a cold/hot/purified water valve 1015 for selectively supplying purified water to the purified water passage 20 and the hot water passage 21 may be installed at a branch point of the purified water passage 20 and the hot water passage 21. In addition,

In addition, a safety device 1025, such as a device for preventing backflow and the like, may be installed in the hot water passage 21. In addition, a safety valve 1016 that discharges steam may be installed in the hot water module 30. The steam of the hot water module 30 may be drained toward the drain 90b through a connected passage 15.

Meanwhile, a water discharge valve 1018 is disposed in the water discharge passage 13 to supply purified water, cold water, and hot water, which flow toward the water discharge part 200, to the water discharge part 200 or may block the water.

In addition, the drain passage 14 may be branched from the water discharge passage 13, or a drain valve 1017 may be disposed in the drain passage 14 to discharge purified water, cold water, hot water, and raw water toward the drain 90b.

Meanwhile, for example, each of the water discharge valve 1018 and the drain valve 1017 may be implemented with a 3-way valve that has one inlet, a first outlet and a second outlet that are selectively opened and includes an actuator that selectively opens and closes the two outlets. In this case, the first outlet may be connected to the water discharge part 200, and the second outlet may be connected to the drain 90b.

Meanwhile, raw water is supplied through the water supply passage 11 which is connected to a water source such as a water pipe, a water tank, or an underground pipe. A decompression valve 1010 is installed in the water supply passage 11, and the raw water passes through the decompression valve 1010 such that the pressure is reduced to a set pressure.

Further, the raw water, from which foreign substances are removed while passing through the filter 10, is converted into purified water. The purified water flows along the purified water passage 20. Further, the water may be branched into cold water-purified water and hot water.

First, the purified water branched into the cold water-purified water is branched again into cold water and purified water, and purified water or cold water may be supplied to a user through the water discharge part 200 based on the operation of the cold water module 40 by a user's operation of selecting purified water or cold water.

When a user requests discharge of cold water, the purified water passes through a cooling coil inside the cold water module 40. The water flowing along the cooling coil is heat-exchanged with a coolant within the cold water module 40 and then is cooled. To this end, the coolant is continuously cooled to maintain a set temperature. For reference, a compressor may be driven to cool the coolant. The driving of the compressor may be determined by a cold water temperature sensor provided in the cold water module 40. Thus, the coolant may be always maintained at the preset temperature, and to this end, the driving of the compressor may be controlled. The compressor may be adjusted in frequency to correspond to a load that is required for an inverter compressor and adjusted in cooling capacity. That is, the compressor may be driven by an inverter control to cool the coolant with optimal efficiency.

Meanwhile, when a user requests discharge of hot water, the water may be heated to a set temperature while passing through the hot water module 30. The hot water module 30 may be heated by an induction heating method, and to this end, an output of a working coil provided in the hot water module 30 may be adjusted. The purified water passing through the hot water module 30 may be heated at the set temperature. The hot water heated while passing through the hot water module 30 may flow toward the water discharge part 200.

Meanwhile, the sterilization passage 71 has a first side branched from the water supply passage 11, and a second side connected to the filter 10. A sterilization module 70 configured to heat water passing through the sterilization passage 71 and a flow control valve 1013 configured to adjust an amount of water flowing into the sterilization passage 71 may be disposed in the sterilization module 70.

Meanwhile, a feed valve 1012 for selectively supplying the raw water to the water supply passage 11 or the sterilization passage 71 may be disposed at a position where the sterilization passage 71 is branched from the water supply passage 11.

Meanwhile, the water supply passage 11 may include a first water supply passage 11a connecting the water source and the feed valve 1012, and a second water supply passage 11b connecting the feed valve 1012 and the filter 10.

Meanwhile, the sensing passage 12 described above may be branched from the first water supply passage 11a at a front end of the feed valve 1012. A sensing valve 1014 configured to open and close the sensing passage 12 and a backflow prevention device 1020 configured to prevent the backflow of raw water may be disposed in the sensing passage 12.

The controller 60 may close the feed valve 1012 and open the sensing valve 1014 to control the raw water to be supplied to the water quality measurement unit 50 through the sensor passage 14.

After measuring the water quality of the raw water, the controller 60 may close the water discharge valve 1018 and open the drain valve 1017 to discharge the raw water, which is measured by the water quality measurement unit 50, toward the drain 90b.

The controller 60 may open the feed valve 1012 toward the purified water passage 20 and close the sensing valve 1014 to control purified water to be supplied to the water quality measurement unit 50.

In addition, the controller 60 may close the water discharge valve 1018 and open the drain valve 1017 to perform a rinse operation by controlling the purified water, having passed through the water quality measurement unit 50, to be discharged toward the drain 90.

Then, the controller 60 may control the valves in the same manner to supply the purified water to the water quality measurement unit 50 for quality measurement of the purified water. Accordingly, it is possible to remove the effect of raw water, and the water quality of the purified water may be accurately measured by the same water quality measurement unit 50.

As described above, the water quality measurement unit 50 includes the turbidity sensor module 55 which is an optical sensor.

FIG. 8 is a diagram referred to in the description of optical turbidity sensing using light.

Referring to FIG. 8, water flows into a chamber 730 through an inlet 740, and the water inside the chamber 730 is discharged through an outlet 750. A light source 710 including light sources and a light receiver 720 configured to receive light are disposed to face each other on the left side or the right side of the chamber 730.

The turbidity sensor module 55 may emit light to some of the raw water or the purified water, and may sense turbidity based on a received scattered light pattern. For example, the turbidity sensor module 55 may detect the scattered light emanating from a visible light laser source and reflected and dispersed by floating substances in water, and output it as a signal value.

Scattered light increases in proportion to the amount of particles in a fluid, and may also be affected by external noise generated by the external environment such as particle behavior or fluid state (physical environment such as formation of bubbles or vortices), vibration, and the like. In addition, in order to measure the turbidity in a low-concentration section more accurately, the virtual impactor 900 is placed at the front end of the turbidity sensor module 55, thereby reducing the flow rate of a fluid flowing into the turbidity sensor module 55, and increasing the accuracy of sensed turbidity values.

FIGS. 9 to 11 are diagrams referred to in the description of a virtual impactor according to an embodiment of the present disclosure.

Referring to FIG. 9, a virtual impactor 900 is disposed at a front end of the turbidity sensor module 55.

The virtual impactor 900 may pass only some of the water flowing into the water quality measurement unit 50, thereby reducing a flow amount of water flowing into the turbidity sensor module 55.

For example, when 100% flow rate of 1.0 to 1.3 L/min of cold water/purified water or 0.4 L/min of hot water is introduced into the virtual impactor 900, the virtual impactor 900 sends only about 10% to 20% flow rate (0.1 to 0.26 L/min of cold water/purified water, 0.04 to 0.08 L/min of hot water) to the turbidity sensor module 500. In addition, the virtual impactor 900 may discharge 80% to 90% flow rate to the outside. In FIG. 9, an example is illustrated in which about 40% to 45% flow rate (0.4 to 0.59 L/min of cold water/purified water, 0.16 to 0.18 L/min of hot water) is discharged through each of the left and right sides.

In addition, contaminants, contained in water flowing into the virtual impactor and the water quality measurement unit 50, are classified by size, thereby increasing the turbidity of water flowing into the turbidity sensor module 55. Accordingly, the flow rate of a measurement target may be reduced, and particle concentration may increase, such that a turbidity value increases and the accuracy and reliability in measurement by the turbidity sensor module 500 are expected to increase.

The virtual impactor 900 may filter out particles larger than or equal to a specific size and discharge the particles to the outside, or may allow them to flow into the turbidity sensor module 55.

The virtual impactor 900 may separate and capture submersible particles by using inertial force for each particle diameter. For example, if a particle size is small, the particle escapes through the outlet of the virtual impactor 900, and if a particle size is large, it cannot escape through the outlet and may be captured.

As described above with reference to FIGS. 2 to 6, an impactor drain passage 14a may be connected to the drain passage 14, and may discharge water and/or contaminants that are prevented from flowing into the turbidity sensor module 55 by the virtual impactor 900. An impactor discharge valve V4 is disposed in the impactor drain passage 14a, and the impactor discharge valve V4 may open and close the impactor drain passage 14a.

According to an embodiment of the present disclosure, by installing the virtual impactor 900 in series or in parallel in multiple stages, turbidity values may be measured for each particle diameter. In addition, after measurement, a contaminant may be inferred as shown in the example below.
Bacteria: 1 μm ↓
Biofilm fragment: 1 μm to 50 μm
Substance that causes rust: 50 μm ↑

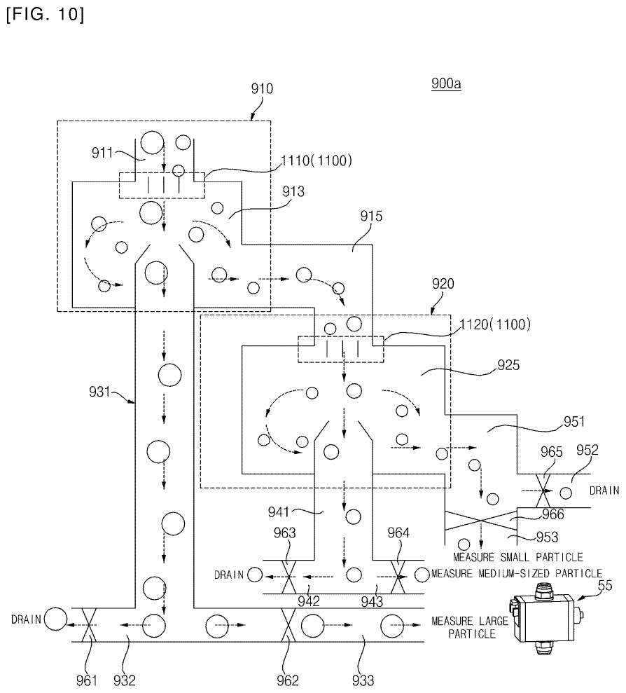

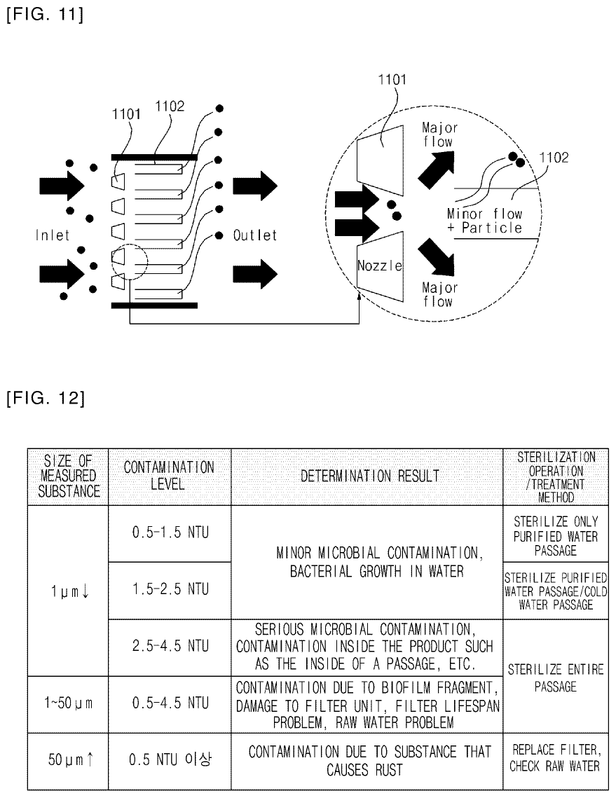

FIG. 10 is a diagram illustrating an example of a virtual impactor for analyzing contaminants by classifying them for each particle diameter, and FIG. 11 is a diagram illustrating an impactor nozzle, and FIGS. 10 and 11 illustrate a multi-stage virtual impactor 900a and an impactor nozzle 1100.

Referring to FIGS. 10 and 11, the virtual impactor 900a may be provided as a multi-stage impactor including impactors 910 and 920 that separate particles of different sizes.

The virtual impactor 900a may classify particles by size using the inertial force of particles that move according to a flow in a passage structure that suddenly changes. In this case, the size of the classified particles may be determined depending on conditions such as a nozzle size and flow rate, etc.

The impactors 910 and 920 include impactor nozzles 1110 and 1120, respectively, and a plurality of slits 1101 are formed in each of the impactor nozzles 1110 and 1120. In order to separate particles by inertial force, a flow rate greater than or equal to a predetermined size is required when the flow is introduced. To this end, as a significant differential pressure is applied when using a single nozzle, it is preferable to provide a multi-nozzle by forming the plurality of slits 1101 in order to minimize the differential pressure. By forming the plurality of slits 1101, the differential pressure inside and outside the slit 1101 is reduced. The differential pressure may be reduced in proportion to the number of slits 1101.

Meanwhile, the impactor nozzles 1110 and 1120 may include an internal passage 1102 through which contaminant particles flow, such as microorganisms introduced through the slit 1101. The internal passage 1102 defines an internal space of the impactor nozzles 1110 and 1120. The contaminant particles may flow through the internal passage 1102, and a fluid, from which the contaminant particles are removed, may flow in an external space of the internal passage 1102.

In the impactor nozzles 1110 and 1120, the slits 1110 and the like are formed differently so that particles of different sizes can be classified.

The virtual impactor 900a may include a first stage impactor 910 configured to separate a first particle, which is a largest particle, from water introduced through an inlet 1011, and a second stage impactor 920 configured to separate a second particle which is smaller than the first particle and a third particle which is a smallest particle. For example, the first stage impactor 910 may separate the first particle, such as a substance than causes rust, etc., which is greater than 50 μm. The second stage impactor 920 may separate the second particle which is a medium-sized particle of 1 μm to 50 μm, such as a biofilm fragment, etc., and a third particle, such as bacteria, etc., which is smaller than 1 μm.

In addition, the virtual impactor 900a may further include a first impactor passage 931 through which the first particle separated by the first stage impactor 910 flows, a second impactor passage 941 through which the second particle separated by the second stage impactor 920 flows, and a third impactor passage 951 through which the third particle separated by the second stage impactor 920 flows.

A fluid from which the first particle is separated by the first stage impactor 910 passes through a connection passage 915 in the internal space 913 of the first stage impactor 910 to flow into the second stage impactor 920. In addition, a fluid from which the second particle is separated by the second stage impactor 920 may flow into the third impactor passage 951 in the internal space 925 of the second stage impactor 920.

The virtual impactor 900a may include a plurality of valves 961, 962, 963, 964, 965, and 966 and may control the flow of a fluid and contaminant particles. The virtual impactor 900a may further include a first impactor valve 961 disposed between the first impactor passage 931 and the impactor drain passage 14a and a second impactor valve 962 disposed between the first impactor passage 931 and the turbidity sensor module 55. The first impactor passage 931 and the impactor drain passage 14a are connected to a first drain connection passage 932, and the first impactor valve 961 may be disposed in the first drain connection passage 932. The first impactor passage 931 and the turbidity sensor module 55 may be connected to a first sensing connection passage 933, and the second impactor valve 962 may be disposed in the first sensing connection passage 933.

The virtual impactor 900a may further include a third impactor valve 963 disposed between the second impactor passage 941 and the impactor drain passage 14a, and a fourth impactor valve 964 disposed between the second impactor passage 941 and the turbidity sensor module 55. The second impactor passage 941 and the impactor drain passage 14a are connected to the second drain connection passage 942, and the third impactor valve 963 may be disposed in the second drain connection passage 942. The second impactor passage 941 and the turbidity sensor module 55 may be connected to the second sensing connection passage 943, and the fourth impactor valve 964 may be disposed in the second sensing connection passage 943.

The virtual impactor 900a may further include a fifth impactor valve 965 disposed between the third impactor passage 951 and the impactor drain passage, and a sixth impactor valve 966 disposed between the third impactor passage 951 and the turbidity sensor module 55. The third impactor passage 951 and the impactor drain passage 14a are connected to the third drain connection passage 952, and the fifth impactor valve 965 may be disposed in the third drain connection passage 952. The third impactor passage 951 and the turbidity sensor module 55 may be connected to the third sensing connection passage 953, and the sixth impactor valve 966 may be disposed in the third sensing connection passage 953.

For example, in order to measure a large particle, the second impactor valve 962 is opened so that the large particles may be introduced into the turbidity sensor module 55, and the third impactor valve 963 and the fifth impactor valve 965 are opened so that medium-sized particles and small particles may be drained. In addition, the first impactor valve 961 is closed so that no large particles are drained, and the fourth impactor valve 964 and the sixth impactor valve 966 are closed so that no medium-sized particles and no small particles are introduced into the turbidity sensor 55.

According to an embodiment of the present disclosure, based on water quality data measured by the water quality measurement unit 50, a sterilization operation may be performed for each passage region by allowing hot water, discharged from the sterilization module 70 described above, to flow to different passage regions.

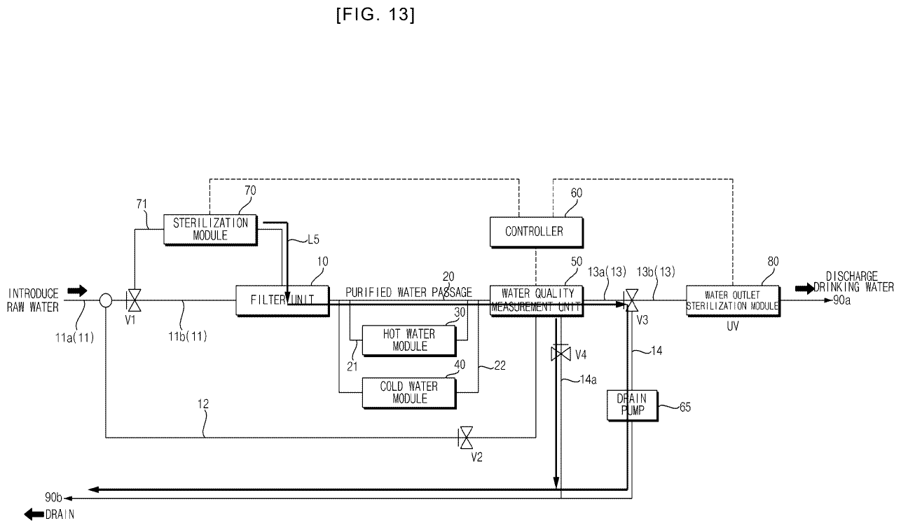

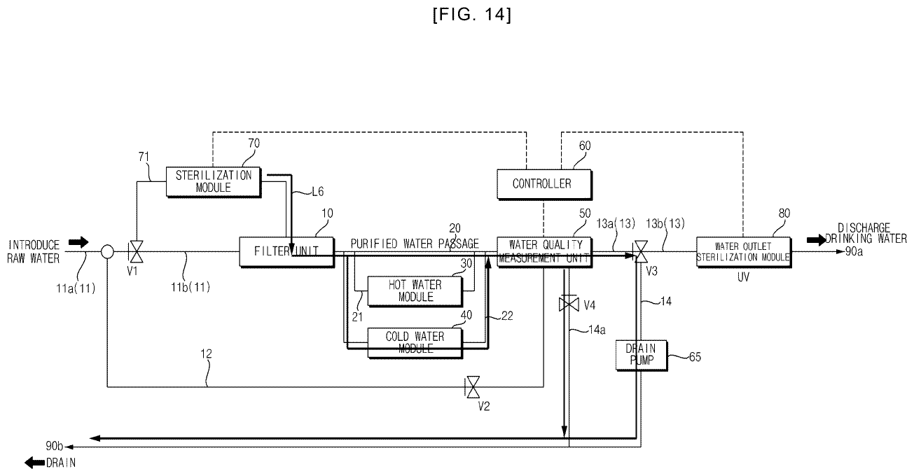

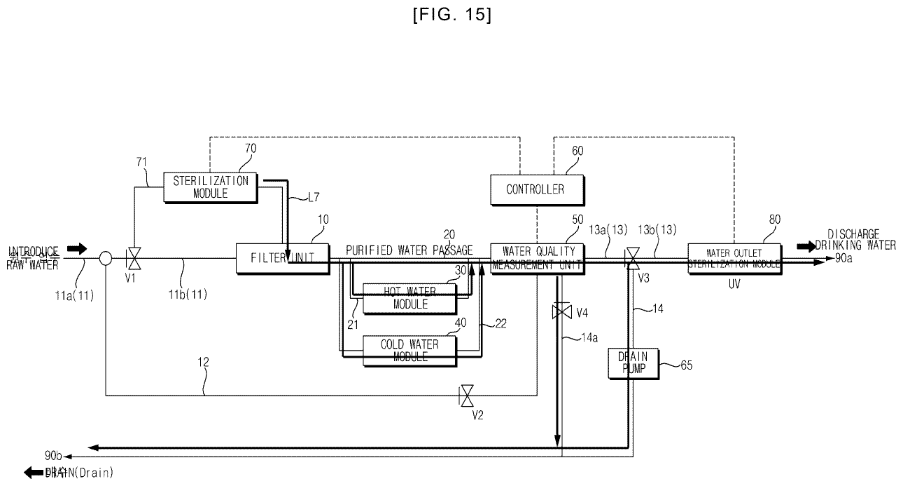

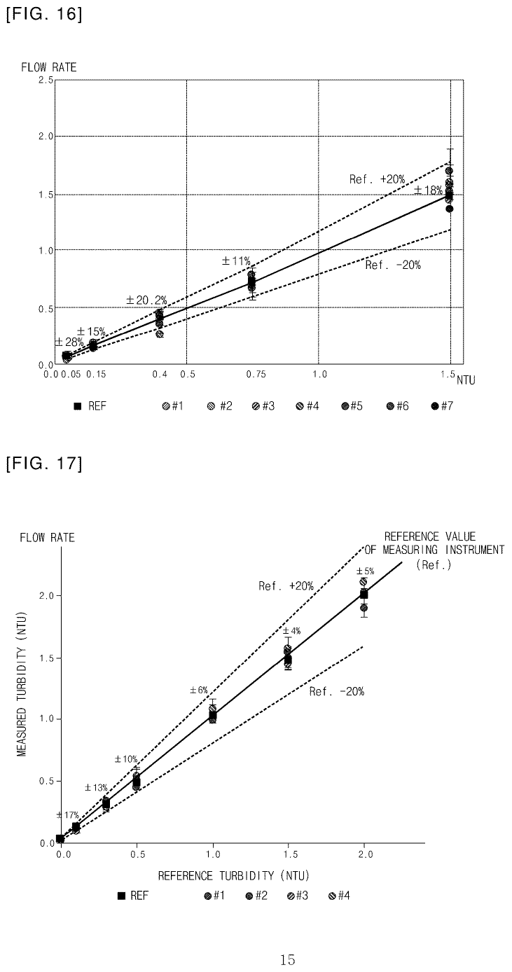

FIG. 12 is a diagram referred to in the description of hygiene management standards for a water dispensing apparatus according to an embodiment of the present disclosure. FIG. 12 is a diagram illustrating hygiene management standards which are set based on contamination level reference values for the size of each measured contaminant, and an example of a sterilization operation performed for each passage section.

Contamination levels can be classified into criteria determined based on drinking water quality standards in each country including the Republic of Korea and the like. In addition, the criteria for classifying contamination levels can be determined based on the drinking water turbidity quality standards of each country including the Republic of Korea and the like. For example, the contamination levels can be classified based on 0.1, 0.3, and 0.5 Nephelometric Turbidity Units (NTU).

Referring to FIG. 12, if a contamination level is less than 0.5 NTU, the controller 60 determines that the level is normal and performs a normal water discharging mode, and if a contamination level is above the level, the controller 60 stops discharging water and performs a sterilization operation by identifying a passage section corresponding to each contamination level. A sterilization algorithm according to an embodiment of the present disclosure is more efficient in that sterilization regions are divided, compared to an existing sterilization algorithm which performs full sterilization without dividing passage regions.

If a contamination level is less than (or less than or equal to) 0.5 NTU, the controller 60 determines that the contamination level is level zero (0), if a contamination level is greater than or equal to (or exceeds) 0.5 NTU and less than (or equal to) 1.5 NTU, the controller 60 determines that the contamination level is level 1, if a contamination level is greater than or equal to (or exceeds) 1.5 NTU and less than (or equal to) 2.5 NTU, the controller 60 determines that the contamination level is level 2, and if a contamination level is greater than or equal to (or exceeds) 2.5 NTU and less than (or equal to) 4.5 NTU, the controller 60 determines that the contamination level is level 3. In the present disclosure, “greater than or equal to” and “exceeds” may be used interchangeably, and “less than” and “less than and equal to” may be used interchangeably.

Generally, a contamination level of 0.5 NTU or more may be determined as a level that requires sanitary management. If water is not used for a long time and is left stagnant for an extended period of time, and when affected by formation of an internal biofilm, and a contamination level increases to reach a level of 2.5 NTU, there is a concern that bacteria may be detected, such that a reference contamination level may be set to 2.5 NTU as a higher contamination level. The level of 2.5 NTU may be set as level 3.

If turbidity of the third particle (e.g., less than 1 μm) is less than a first reference value (e.g., 0.5 NTU), the contamination level is low at zero, such that the controller 60 may control water to be discharged normally without performing an additional sterilization operation.

By contrast, if water quality data measured for the third particle by the water quality measurement unit 50 is greater than or equal to the first reference value, the controller 60 may control hot water, discharged from the sterilization module 70, to sterilize the purified water passage 20.

If the water quality data measured for the third particle by the water quality measurement unit 50 is greater than or equal to a second reference value (e.g., 1.5 NTU) that is greater than the first reference value, and is less than a third reference value (e.g., 2.5 NTU) that is greater than the second reference value, the controller 60 may control hot water, discharged from the sterilization module 70, to sterilize the purified water passage 20 and the cold water passage 22.

Alternatively, if the water quality data measured for the third particle by the water quality measurement unit 50 is greater than or equal to the second reference value (e.g., 1.5 NTU) that is greater than the first reference value, and is less than the third reference value (e.g., 2.5 NTU) that is greater than the second reference value, the controller 60 may control hot water, discharged from the sterilization module 70, to sterilize the purified water passage 20 and the water discharge passage 13.

The third reference value is a criterion for classifying minor microbial contamination, bacterial growth in water and serious microbial contamination, and contamination inside the product such as the inside of a passage, in which if a contamination level is above the third reference value, it is required to apply enhanced hygiene management.

If the water quality data measured for the third particle by the water quality measurement unit is greater than or equal to the third reference value that is greater than the second reference value, the controller 20 may control hot water, discharged from the sterilization module 70, to sterilize the entire passage.

Meanwhile, if a contaminant particle has a large size, enhanced hygiene management may be applied.

The second particle, such as a biofilm fragment, may cause serious contamination inside the product, such as the inside of a passage, and the third particle, such as a substance that causes rust, may cause a serious problem including damage to the filter unit 10, lifespan problem, raw water problem, and the like.

If the water quality data measured for the second particle (e.g., 1 μm to 50 μm) by the water quality measurement unit 50 is greater than or equal to the first reference value, the controller 20 may control hot water, discharged from the sterilization module 70, to sterilize the entire passage.

In addition, if the water quality data measured for the first particle (e.g., greater than or equal to 50 μm) by the water quality measurement unit 50 is greater than or equal to the first reference value, the controller 20 may control the output unit 85 configured to display abnormal water quality information. In addition, the controller 60 may report the situation to a manufacturer and seller, and service technicians of the manufacturer and seller may visit the site and check the raw water. If there is no problem with the raw water, all or part of the filter 10 may be replaced, and if there is a problem with the raw water, you can contact your local water supply management agency.

In the present disclosure, it is possible to select and sterilize a passage according to a contamination level measured by the water quality measurement unit 50. By sterilizing a specific region, it is possible to reduce the time required for sterilization. In addition, if about 15 L of water is used for performing the entire sterilization operation, a required amount of water is reduced to about 8 L when an operation of sterilizing individual passages is performed, such that by reducing the amount of water needed for sterilization, it is possible to efficiently manage and operate the water dispensing apparatus from a long-term perspective.

FIGS. 13 to 15 are diagrams referred to in the description of sterilizing a water dispensing apparatus according to an embodiment of the present disclosure. FIG. 13 is a diagram illustrating an example of sterilization operation for water contamination level 1 of small particles, FIG. 14 is a diagram illustrating an example of sterilization operation for water contamination level 2 of small particles, and FIG. 15 is a diagram illustrating an example of sterilization operation for water contamination level 3 of small particles and water contamination level 1 of medium-sized particles.

Referring to FIGS. 13 to 15, the water dispensing apparatus includes a water supply passage 11 through which raw water supplied from a water source flows, and a filter unit 10 configured to generate purified water by filtering the raw water supplied through the water supply passage 11.

The purified water having passed through the filter unit 10 flows toward the water outlet 90a through the purified water passage 20. The purified water having passed through the filter unit 10 may flow into the water quality measurement unit 50. When the purified water is introduced, the water quality measurement unit 50 may measure water quality of the purified water.

Meanwhile, a sterilization passage 13 may be branched from the water supply passage 11. The sterilization passage 13 may have a first side branched from the water supply passage 11 and a second side connected to the filter unit 10. The sterilization module 70 configured to generate hot water for sterilization is disposed in the sterilization passage 13.

Based on the water quality data measured by the water quality measurement unit 50, the water dispensing apparatus may control hot water, discharged from the sterilization module 70, to flow to different passage regions to perform a sterilization operation for each region. Accordingly, the passage may be efficiently sterilized within a short period of time.

Referring to FIGS. 13 to 15, the water dispensing apparatus includes a water outlet 90a through which the purified water is discharged, a water discharge passage 13 guiding the purified water toward the water outlet 90a, a drain passage 14 which is branched from the water discharge passage 13 between the water quality measurement unit 50 and the water outlet 90a and through which the raw water or the purified water is discharged, and the water discharge passage 13 through which the raw water or the purified water.

A water discharge valve V3 is disposed in the water discharge passage 13, so as to selectively supply the raw water or the purified water to the water discharge passage 13b and the drain passage 14.

In addition, a hot water passage 21 and a cold water passage 22 are branched from the purified water passage 20. A hot water module 30 and a cold water module 40 configured to heat and cool the purified water are disposed in the hot water passage 21 and the cold water passage 22, respectively.

If water quality data measured by the water quality measurement unit 50 is greater than or equal to a first reference value, the hot water discharged from the sterilization module 70 may sterilize the purified water passage 20.

Referring to FIG. 13, in response to contamination level 1 of small particles, the controller 60 may control hot water, discharged from the sterilization module 70, to sterilize the purified water passage 20. Along a fifth line L5, the hot water sterilizes the purified water passage 20 and is drained through the drain passage 14.

In addition, if water quality data measured by the water quality measuring unit 50 is greater than or equal to a first reference value, discharge of water through the water outlet may be stopped. While stopping the discharge of drinking water, the controller 60 may selectively sterilize a passage in response to a contamination level thereof, and then may resume discharge of water.

Meanwhile, if water quality data measured by the water quality measuring unit 50 is greater than or equal to a second reference value that is greater than the first reference value, the purified water passage 20 and the cold water passage 22 (in some examples, the water discharge passage 13) may be sterilized with the hot water discharged from the sterilization module 70.

Referring to FIG. 14, in response to contamination level 2 of small particles, the controller 60 may control hot water, discharged from the sterilization module 70, to sterilize the purified water passage 20 and the cold water passage 22. Along a sixth line L6, the hot water sterilizes the purified water passage 20 and the cold water passage 22 and is drained through the drain passage 14.

If water quality data measured by the water quality measuring unit 50 is greater than or equal to a third reference value that is greater than the second reference value, the entire passage may be sterilized with the hot water discharged from the sterilization module 70. The entire passage refers to the entire passage that can be sterilized with hot water, and does not mean all of the passages of the water dispensing apparatus. For example, the sensing passage 12 is not inclined in the entire passage during a sterilization operation.

Referring to FIG. 15, in response to contamination level 3 of small particles or contamination level 2 of medium-sized particles, the controller 60 may control hot water, discharged from the sterilization module 70, to sterilize the purified water passage 20, the hot water passage 21, the cold water passage 22, and the water discharge passage 13. Along the sixth line L6, the hot water sterilizes the purified water passage 20, the hot water passage 21, and the cold water passage 22, and some of the hot water is drained through the drain passage 14. In addition, some of the hot water sterilizes the water discharge passage 13 and is drained through the water outlet 90a.

FIGS. 16 and 17 are diagrams referred to in the description of distribution of measured turbidity values, in which FIG. 16 is a diagram illustrating distribution of measured turbidity values/flow rates, and FIG. 17 is a diagram illustrating reference turbidity and measured turbidity values at a given flow rate.

Referring to FIGS. 16 and 17, as the turbidity value increases, the measured distribution decreases. In addition, as a flow rate decreases, the measured distribution decreases (+18%→±4% at 1.5 NTU), and as the turbidity value increases, the measured distribution decreases (±10-17% at 0.5 NTU or less and ±5-6% at 1.0 NTU, with a flow rate of 0.3 L/min).

Accordingly, in the present disclosure, by providing the virtual impactor 900a at a front end of the turbidity sensor module 55, the flow rate of water to be measured that flows into the turbidity sensor module 55 may be reduced, and the turbidity value may increase. By performing measurement on a sample in which the flow rate is reduced and the turbidity value increases, the turbidity may be measured more accurately and precisely.

It will be apparent that, although the preferred embodiments have been illustrated and described above, the present disclosure is not limited to the above-described specific embodiments, and various modifications and variations can be made by those skilled in the art without departing from the gist of the appended claims.. Thus, it is intended that the modifications and variations should not be understood independently of the technical spirit or prospect of the present disclosure.

1. A water dispensing apparatus comprising:
a water supply passage through which raw water, supplied from a water source, flows;
a filter configured to generate purified water by filtering the raw water supplied through the water supply passage;
a purified water passage through which the purified water, having passed through the filter, flows; and
a water quality measurement unit connected to the purified water passage and configured to measure water quality of the purified water,
wherein the water quality measurement unit comprises a turbidity sensor module configured to measure turbidity of water, and a virtual impactor disposed at a front end of the turbidity sensor module.

2. The water dispensing apparatus of claim 1, wherein the virtual impactor is configured to reduce a flow rate of water flowing into the turbidity sensor module, by passing only some of water that flows into the water quality measurement unit.

3. The water dispensing apparatus of claim 1, wherein the virtual impactor is configured to classify by size contaminants contained in the water flowing into the water quality measurement unit, to increase turbidity of the water flowing into the turbidity sensor module.

4. The water dispensing apparatus of claim 1, further comprising:
a drain passage through which the raw water or the purified water is drained;
an impactor drain passage which is connected to the drain passage, and through which water not introduced by the virtual impactor into the turbidity sensor module is discharged; and
an impactor discharge valve disposed in the impactor drain passage.

5. The water dispensing apparatus of claim 4, further comprising:
a water outlet through which the purified water is discharged; and
a water discharge passage guiding the purified water to the water outlet,
wherein the drain passage is branched from the water discharge passage between the water quality measurement unit and the water outlet.

6. The water dispensing apparatus of claim 5, further comprising a water discharge valve selectively supplying the raw water or the purified water to the water discharge passage and the drain passage.

7. The water dispensing apparatus of claim 4, wherein the virtual impactor is provided as a multi-stage impactor including impactors that separate particles of different sizes.

8. The water dispensing apparatus of claim 7, wherein the virtual impactor further comprises:
a first stage impactor configured to separate a first particle which is a largest particle;
a second stage impactor configured to separate a second particle which is smaller than the first particle and a third particle which is a smallest particle;
a first impactor passage through which the first particle separated by the first stage impactor flows;
a second impactor passage through which the second particle separated by the second stage impactor flows; and
a third impactor passage through which the third particle separated by the second stage impactor flows.

9. The water dispensing apparatus of claim 8, wherein the virtual impactor further comprises:
a first impactor valve disposed between the first impactor passage and the impactor drain passage;
a second impactor valve disposed between the first impactor passage and the turbidity sensor module;
a third impactor valve disposed between the second impactor passage and the impactor drain passage;
a fourth impactor valve disposed between the second impactor passage and the turbidity sensor module;
a fifth impactor valve disposed between the third impactor passage and the impactor drain passage; and
a sixth impactor valve disposed between the third impactor passage and the turbidity sensor module.

10. The water dispensing apparatus of claim 7, further comprising:
a sterilization passage having a first side branched from the water supply passage and a second side connected to the filter side; and
a sterilization module provided in the sterilization passage and configured to heat water passing through the sterilization passage,
wherein a sterilization operation is performed for each passage region by controlling the hot water, discharged from the sterilization module, to flow to different passage regions based on water quality data measured by the water quality measurement unit.

11. The water dispensing apparatus of claim 10, wherein in response to water quality data measured for the third particle by the water quality measurement unit being greater than or equal to a first reference value, the water dispensing apparatus is configured to sterilize the purified water passage with the hot water discharged from the sterilization module.

12. The water dispensing apparatus of claim 11, further comprising:
a hot water passage having one side branched from the purified water passage;
a hot water module provided in the hot water passage and configured to heat purified water passing through the hot water passage;
a cold water passage having one side branched from the purified water passage; and
a cold water module provided in the cold water passage and configured to cool purified water passing through the cold water passage,
wherein in response to water quality data measured for the third particle by the water quality measurement unit being greater than or equal to a second reference value that is greater than the first reference value, the water dispensing apparatus is configured to sterilize the purified water passage and the cold water passage with the hot water discharged from the sterilization module.

13. The water dispensing apparatus of claim 12, wherein in response to water quality data measured for the third particle by the water quality measurement unit being greater than or equal to a third reference value that is greater than the second reference value, the water dispensing apparatus is configured to sterilize an entire passage with the hot water discharged from the sterilization module.

14. The water dispensing apparatus of claim 11, wherein in response to water quality data measured for the third particle by the water quality measurement unit being greater than a second reference value that is greater than the first reference value, the water dispensing apparatus is configured to sterilize the purified water passage and the water discharge passage with the hot water discharged from the sterilization module.

15. The water dispensing apparatus of claim 11, wherein in response to the water quality data measured for the second particle by the water quality measurement unit being greater than or equal to the first reference value, the water dispensing apparatus is configured to sterilize the entire passage with the hot water discharged from the sterilization module.

16. The water dispensing apparatus of claim 11, further comprising an output unit which, in response to water quality data measured for the first particle by the water quality measurement unit being greater than or equal to the first reference value, is configured to display abnormal water quality information.

17. The water dispensing apparatus of claim 1, further comprising a sensing passage which is branched from the water supply passage, and through which the raw water flows,
wherein the water quality measurement unit is configured to measure water quality of the purified water in response to the purified water being introduced through the purified water passage, and configured to measure water quality of the raw water in response to the raw water being introduced through the sensing passage.

18. The water dispensing apparatus of claim 17, wherein upon measuring the water quality of the raw water, the water quality measurement unit is configured to measure the water quality of the purified water after performing a rinse operation in which the purified water passes through the water quality measurement unit one or more times.

19. The water dispensing apparatus of claim 17, further comprising:
a switching valve configured to supply the raw water to the water supply passage or the sterilization passage; and
a sensing valve configured to open and close the sensing passage.
